(19) United States
(12) Patent Application Publication
(10) Pub. No.: US 2008/0281200 A1
(43) Pub. Date: Nov. 13, 2008

(54) Elevated coupling liquid temperature during HIFU treatment method and hardware

(75) Inventors: Dan Voic, Cedar Grove, NJ (US); Ronald R. Manna, Valley Stream, NY (US)

Correspondence Address: COLEMAN SUDOL SAPONE, P.C., 714 COLORADO AVENUE, BRIDGE PORT, CT, 06605-1601, US

(73) Assignee: MISONIX, INCORPORATED, Farmingdale, NY (US)

(21) Appl. No.: 11/801,912

(22) Filed: May 10, 2007

(51) Int. Cl. A61B 17/00

(52) U.S. Cl. 600/439

(57) ABSTRACT

A medical procedure utilizes a high-intensity focused ultrasound instrument having an applicator surface, a liquid-containing bolus or expandable chamber acting as a heat sink, and a source of ultrasonic vibrations, the applicator surface being a surface of a flexible wall of the bolus, the source of ultrasonic vibrations being in operative contact with the bolus. The applicator surface is placed in contact with an organ surface of a patient, the source is energized to produce ultrasonic vibrations focused at a predetermined focal region inside the organ, and a temperature of liquid in the bolus is controlled while the applicator surface is in contact with the organ surface to control temperature elevation in tissues of the organ between the focal region and the organ surface to necrose the tissues to within a desired distance from the organ surface.

### BACKGROUND OF THE INVENTION

This invention relates to medical treatment procedures and apparatus using high-intensity focused ultrasound. More particularly, this invention relates to such procedures and apparatus for inducing controlled tissue necrosis.

High Intensity Focused Ultrasound (“HIFU”) devices for use in various surgical procedures have been described in medical literature since the late 1940's. These devices use the same type of energy source as is found in SONAR transmitters or more recently, Diagnostic Ultrasound Scanners. However, instead of transmitting these waves through the body as a collimated beam, HIFU transmitters instead focus the acoustic energy to a theoretical point distal from the transducer/tissue interface much the same as a magnifying glass focuses light beams. The point at which the acoustic energy intensity is greatest is called the focal point. If the energy intensity is great enough at this point, the tissue will begin to heat. As the energy level is increased further, the tissue will heat to the point where cell death occurs, called the necrosis point. After this, the tissue is unviable and will die, even if the energy source is turned off. In this manner, tissue can be destroyed deep inside the body without disturbing the intervening tissue, where the acoustic intensity is below that where necrosis will occur.

There have been several implementations of this theory described in the prior art. However, almost all of these implementations or embodiments have the requirement of creating an acoustically efficient coupling between the acoustic wave generator and the tissue itself. In addition, several embodiments of HIFU devices use a moving piezoelectric transducer face to aim the focal point at different targets within the body. The transducer is moved by stepping motors with digital feedback under control of the main computer. The transducer is placed within a hollow sleeve with an opening through which the acoustic beams emanate. The transducer is free to move longitudinally and rotationally without touching the skin at all. This assembly is called the transducer head 101, shown in FIG. 1.

Since gas presents infinite impedance to acoustic energy, no energy would flow from the transducer face to the body organ in contact with the transducer head if the internal volume remains filled with air. In current embodiments, a flexible membrane is placed over the transducer head and sealed. The internal volume of the head is filled with water that provides acoustic coupling between the piezoelectric transducer and the body organ itself. Since most mammalian bodies are water based, the acoustic impedance between the transducer coupling water and the body is low, thereby providing efficient transmission of the acoustic waves from the piezoelectric element and the target tissue.

The liquid used must have distinct properties, such as, it must be gas free or nearly so, it must be sterile if it is to be introduced in surgical procedures under the skin and it must be readily available and inexpensive. U.S. patent application Ser. No. 11/355,055 entitled “Liquid Processing and Handling Apparatus and Associated Method for Use in Medical Procedures,” explains these requirements in some detail.

When the HIFU treatment is designed to target a volume of tissue significantly below the surface of the organ or skin, the liquid coupling fluid is generally kept below body temperature during the entire time of the HIFU energy application. This is because the tissue to membrane interface must be cooled to prevent the heat created at the focal zone of the acoustic energy from conducting back to the interface and causing unwanted tissue damage. To date, all known HIFU systems include a liquid handling system or other device to keep the interface between the coupling fluid or gel and the surface tissue cool.

However, there are new devices coming to the market which use HIFU energy to create an ablation zone which extends from the focal zone of the acoustic energy all the way back to the surface of the organ being treated. In these devices, a treated tissue/membrane interface is desired. Several impediments to the creation of these lesions are encountered when current HIFU hardware systems are employed for this scheme of treatment.

First, the cooled fluid used for coupling the acoustic energy emitted from the transducer to the tissue keeps the interface below the necrosis temperature by simple conductive cooling. Therefore, the lesion cannot extend all the way back to the surface and in fact is limited to a few millimeters from the surface due to the high thermal sink presented by the bolus filled with cool fluid.

One scheme to counter this is to increase the acoustic energy level of the transducer output to a point which overcomes the cooling capacity of the bolus. When this has been tried, the acoustic intensity at the focal point is so high that unwanted side effects occur, such as cavitation at the focal zone and lesion volumes which are much larger than desired, resulting in high collateral tissue damage. This prevents the use of the device near important structures like nerve bundles or bile ducts, which need to be spared.

It is therefore desired to create a method and hardware which will allow application of HIFU energy into a tissue volume and allow a controlled lesion to be created from the focal zone all the way back the organ surface consistently.

### OBJECTS OF THE INVENTION

One object of this invention is to provide a surgical method to allow clinicians to create a contiguous lesion from the focal zone of a HIFU transducer all the way back to the surface of the organ being treated.

Another object of this invention is to describe a hardware embodiment that will provide a means to allow a surgeon to treat a contiguous lesion from the focal point of a HIFU transducer all the way back to the surface of the organ being treated.

These and other objects of the invention will be apparent from the drawings and descriptions herein. Although every object of the invention is attained in at least one embodiment of the invention, there is not necessarily any embodiment which attains all of the objects of the invention.

### SUMMARY OF THE INVENTION

This invention relates to a scheme of using elevated liquid temperatures during High Intensity Focused Ultrasound (HIFU) procedures and the equipment and techniques necessary for its implementation and use.

During the use of existing HIFU systems, a coupling fluid is provided in a pool or pumped into a bolus, which allows the fluid to surround the ultrasonic transducer. This coupling fluid is generally degassed, sterile water. Since most of these systems target tissue below the organ surface, this liquid is kept significantly below body temperature to enhance acoustic coupling and to prevent necrosis of the tissue at the surface.

In the present invention, a method is proposed where the temperature of the fluid is kept below the body temperature for the majority of the time of the HIFU treatment. This allows the lesion to develop normally at the acoustic focal point, as is standard practice. As the HIFU energy kept on after the initial lesion is generated, the lesion grows back toward the transducer face due to the thermal conduction of the tissue between the lesion and the transducer. Tissue is generally not equally ablated distal to the initial lesion due to a phenomenon known as Acoustic Shadowing. This phenomenon is well known to the art.

However, the lesion will have a maximum penetration distance proximal to the focal zone because of the cooling effect of the coupling fluid on the tissue it comes into contact with, separated only by the thin elastomeric bolus. This point will be the point where the heat flux into the tissue from conduction from the focal zone equals the heat removed due to the thermal sink properties of the fluid bolus through the intervening tissue.

If one can remove the heat sink potential of the bolus, then the lesion may grow back to the tissue surface itself.

This invention involves the control of the coupling fluid temperature during and after HIFU treatment.

One method of operation which has been developed is to treat the organ with HIFU energy in the prior art method for a good deal of the treatment time. After a specific time, or when diagnostic visualization of the treatment zone reveals that the lesion has grown back to within the desired distance from the surface, the HIFU energy will be suspended. Then the coupling fluid temperature will be increased to approximately 55 to 70° C. The bolus will be left in place until the tissue is necrosed from the surface distally to the lesion created by the HIFU Energy with no intervening viable tissue left. This thermally produced lesion will grow by thermal conduction from the hot bolus to the original HIFU lesion.

In another method embodiment, the bolus fluid will be maintained at temperature loosely around the body or organ temperature point. In yet another embodiment, the temperature may be maintained just below the necrosis point of tissue, generally regarded as 42° C. but not a constant. Then the heat sinking properties of the bolus is reduced and any heat input to the tissue from thermal conduction from the focal zone will increase the temperature of the tissue above necrosis, creating the desired contiguous lesion.

In yet another embodiment, the bolus fluid temperature will be controlled via feedback mechanisms from inputs from sensors such as thermocouples, Infrared Cameras or other such devices known to the art to enable a contiguous lesion to be developed automatically, with minimal input from the surgical team.

The invention includes hardware embodiments that will facilitate the heating of the bolus fluid and the control of the temperature level from a control panel located on the HIFU generator user interface or remotely via an Ethernet or other electronic or radiofrequency communication scheme.

One such embodiment includes an inline heating element which is installed in the fluid line at the input to the HIFU probe housing. Such an element will be sized to provide a temperature rise in the flowing coupling fluid to a temperature desired by the user. A feedback thermocouple may be provided to communicate with the coupling fluid to monitor fluid temperature and to signal a liquid temperature controller to turn the heater on or off or control the electrical energy to the heater with PID control loops in methods known to the art.

In another hardware embodiment, such heaters may be in located in the bolus region itself. This reduces the time lag for bolus fluid heating and also reduces the temperature loss due to heating the tubing runs throughout the probe body.

In another embodiment, the liquid flow through the bolus may be stopped while the heating is taking place. In this way, minimum heat energy is needed to heat the fluid. More importantly, immediate cooling may be accomplished by turning off the heater and turning on the fluid pumping system, which will flood the area with cool fluid and allow cooling of the tissue in cases where an overheat condition is encountered or to control the lesion size and shape more precisely.

Accordingly, a medical procedure in accordance with the present invention utilizes a high-intensity focused ultrasound instrument having an applicator surface, a liquid-containing bolus or expandable chamber acting as a heat sink, and a source of ultrasonic vibrations, the applicator surface being a surface of a flexible wall of the bolus, the source of ultrasonic vibrations being in operative contact with the bolus. The method further comprises placing the applicator surface in contact with an organ surface of a patient, energizing the source to produce ultrasonic vibrations focused at a predetermined focal region inside the organ, and controlling a temperature of liquid in the bolus while the applicator surface is in contact with the organ surface to control temperature elevation in tissues of the organ between the focal region and the organ surface to necrose the tissues to within a desired distance from the organ surface.

Typically, the energizing of the source of ultrasonic vibrations continues for a time period long enough to necrose tissues in a predetermined region internal to the patient. Pursuant to one particular embodiment of the present invention, the temperature of the liquid in the bolus is maintained at a preselected temperature approximately equal to a natural body temperature of the organ at least during an interval subsequent to an initial portion of the time period of focused ultrasound application, and possibly during the entire period of ultrasound application. Pursuant to another particular embodiment of the present invention, the temperature of the liquid in the bolus is maintained at a preselected temperature substantially equal to and less than a necrotizing temperature of the tissues of the organ at least during an interval subsequent to an initial portion of the period of focused ultrasound application, and in some cases during the entire period of ultrasound application.

According to another feature of the present invention, where the instrument includes a liquid flow circuit including the bolus, the controlling of the temperature of the liquid in the bolus includes controlling a rate of liquid flow through the circuit and the bolus. This flow control may be accomplished, for instance, by regulating the speed of a pump or by adjusting a valve. Controlling the rate of liquid flow includes the option of at least temporarily arresting liquid flow through the circuit and the bolus.

Pursuant to a further particular embodiment of the present invention, the controlling of the temperature of the liquid in the bolus includes increasing the temperature of the liquid in the bolus after an initial interval of the ultrasound application. The temperature increase is typically from a first temperature substantially below the necrotizing temperature of the tissues of the organ to a second temperature substantially above the necrotizing temperature. This temperature increase may be instituted after the ultrasound application has been terminated.

Temperature control may be effectuated by heating the liquid by operating a heat source inside the bolus or in a liquid-flow circuit upstream of the bolus.

Where the instrument includes a temperature sensor and a heating element, the controlling of the temperature of the liquid in the bolus may include automatically energizing the heating element in response to a signal from the sensor.

A medical treatment apparatus comprises, in accordance with the present invention, a high-intensity focused ultrasound instrument having an applicator surface, a bolus acting as a heat sink, and a source of ultrasonic vibrations. The applicator surface is in thermal and operative contact with the bolus. The source of ultrasonic vibrations is also in operative contact with the bolus. The apparatus further comprises means for controlling a temperature of liquid in the bolus while the applicator surface is in contact with the organ surface to control temperature elevation in tissues of the organ between the focal region and the organ surface to necrose the tissues to within a desired distance from the organ surface. This desired distance may be zero.

The apparatus may additionally comprise a liquid supply circuit communicating with the bolus for circulating liquid thereto, while the means for controlling includes a heating element disposed in the liquid supply circuit upstream of the bolus. The means for controlling may further include a temperature sensor operatively connected to the heating element. Alternatively, the means for controlling includes a heating element disposed in the bolus.

The means for controlling may include an adjustable rate pump in operative engagement with a liquid supply circuit communicating with the bolus for circulating liquid thereto.

### BRIEF DESCRIPTION OF THE DRAWINGS

FIG. 1 is a schematic elevational view of a typical HIFU transducer head assembly with temperature control components in accordance with the present invention.

FIG. 2A is a diagram of a HIFU transducer head bolus.

FIG. 2B is a graph showing bolus height as a function of liquid level changes.

FIG. 3 is a diagram of a hydraulic circuit for charging the bolus with liquid, also showing temperature control components in accordance with the present invention.

FIG. 4 is a diagram of a portion of the circuit of FIG. 3, constituting a liquid degassing and reservoir subcircuit.

FIG. 5 is a diagram of another portion of the circuit of FIG. 3, constituting a HIFU transducer head purging subcircuit.

FIG. 6 is a diagram of a HIFU transducer liquid circuit configured from the circuit of FIG. 3.

### DETAILED DESCRIPTION

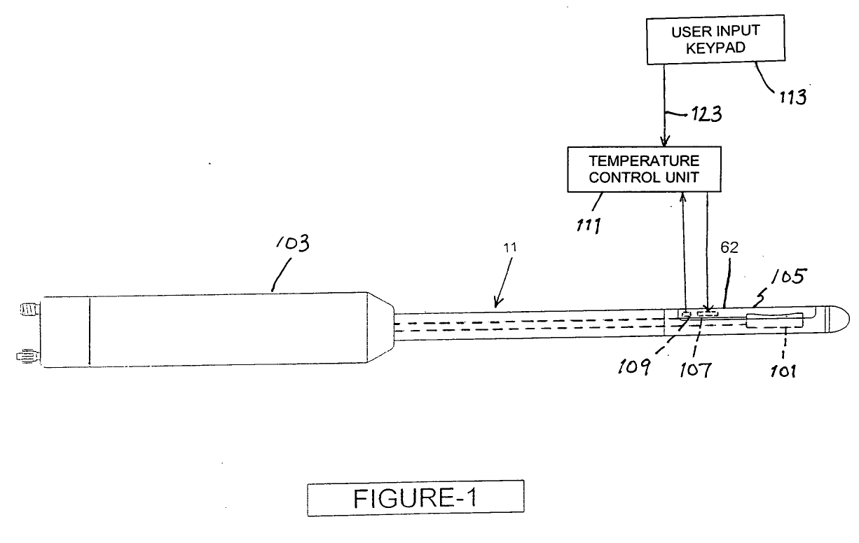

FIG. 1 depicts a high-intensity focused ultrasound probe 11 including a handle or handgrip 103 and a transducer head 101 disposed inside an expandable liquid chamber or bolus 62. The transducer head is a source of ultrasonic vibrations in operative engagement with bolus 62. Probe 11 further includes means for controlling a temperature of liquid in the bolus 62 while an applicator surface 105 (a surface of a flexible wall of the bolus, not separately designated) is in contact with an organ surface of a patient to control temperature elevation in tissues of the organ between a focal region and the organ surface to necrose the tissues to within a desired distance from the organ surface. Modes of operating probe 11 with bolus temperature control are discussed below, after a description of a liquid processing and transport system for the probe.

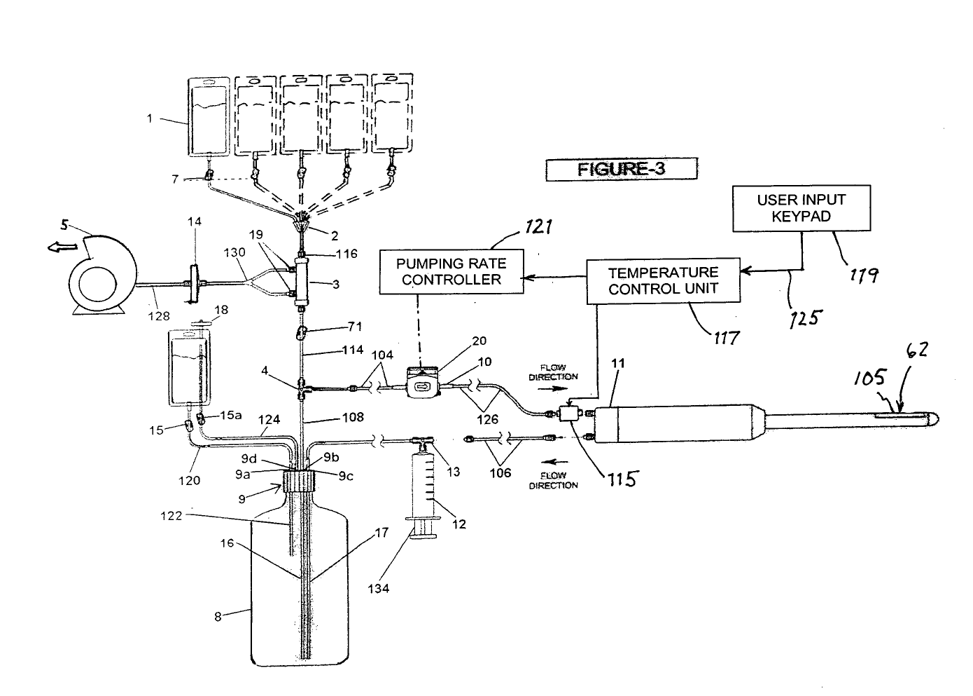

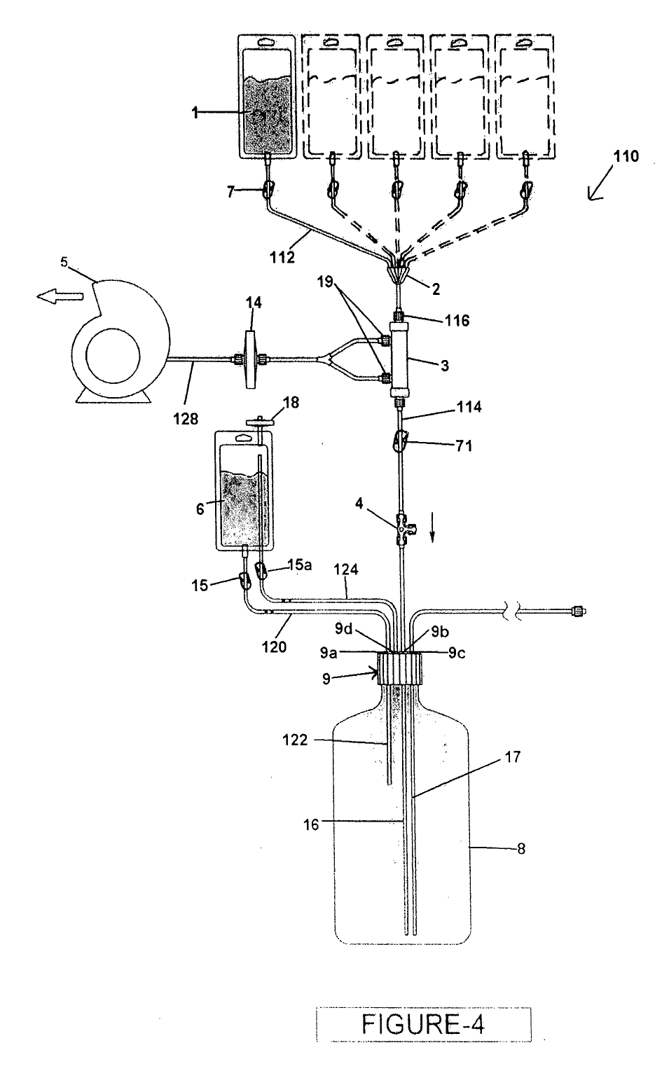

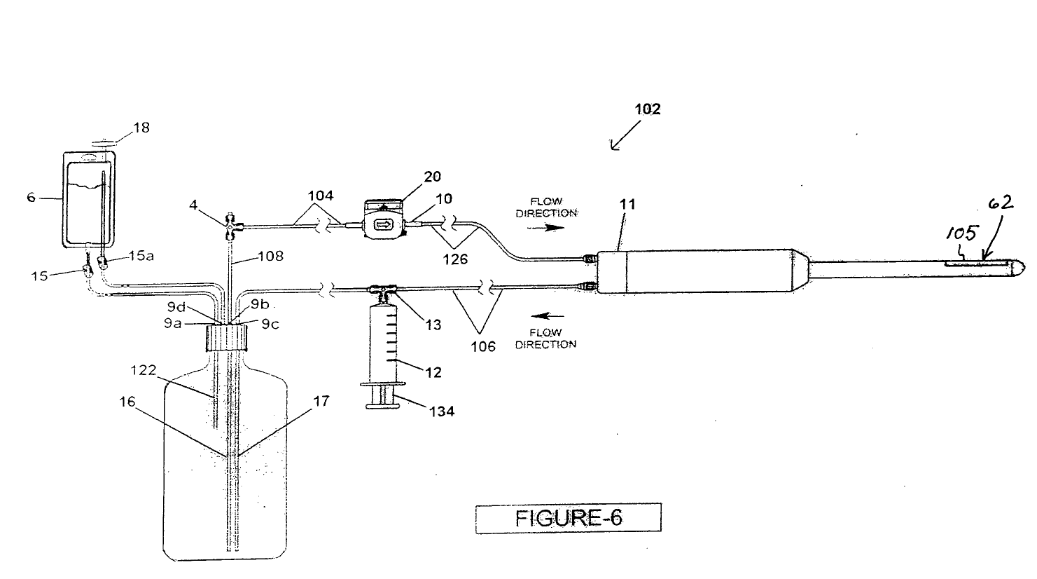

A liquid processing and transport mechanism for a medical instrument such as a high intensity focused ultrasound probe 11 is shown in FIG. 3. In this embodiment, the objective is to construct and load a closed loop pumping system or powered hydraulic circuit 102 (FIG. 6) including the ultrasound probe 11 and a reservoir bottle 8 containing degassed sterile medical irrigation water. More specifically, the closed loop hydraulic circuit 102 includes a liquid manifold such as a three-way valve 4, pump feed tubing 104, a peristaltic pump 20, a pump outlet tube 10, the transducer head assembly or ultrasound probe 11, tubing 106 extending from the probe to reservoir bottle 8, the reservoir bottle 8, and a return tube 108. Hydraulic circuit 102 is loaded with degassed sterile medical irrigation water via an auxiliary hydraulic circuit 110 (FIG. 4) including one or more sterile water supply bags 1, a bag-to-degasser-unit tube 112, a valve or manifold 2, a degasser unit 3, a degasser-to-manifold tube 114, and an overflow bag 6. A top-up syringe 12 may also be part of the system.

All interior and exterior surfaces of the components of the system must be sterilized prior to assembly by steam autoclave, Ethylene Oxide Gas (ETO), gamma irradiation or other means as may be appropriate.

The operating room set-up personnel will assemble the system in the configuration as shown in FIG. 3 using standard luer fittings or other such means of liquid and airtight connections. The one or more sterile water supply bags or bottles 1 serve as a liquid source and may be standard “Water for Irrigation” containers, whether flexible or rigid. As is known in the art, sterile pure water must be used where any portion of the probe 11 is placed in the body under the skin. Such water is readily available in the marketplace and is relatively inexpensive. If a greater volume of liquid is desired than is contained in a single unit, multiple bags or bottles 1 are connected via multi-inlet manifold 2 using standard IV spikes to connect to the sterile water containers. The water supply container or containers 1 must be mounted higher than rigid reservoir container 8 to allow a gravity head to be developed or, alternatively, a pump may be employed. Each water supply bag or bottle 1 should incorporate either a stopcock or a pinchvalve 7 to control outlet flow or the tubing attached to each supply bag or bottle 1 should contain a stopcock or shut-off valve.

A liquid tube 112 from a single supply bag or bottle 1 and additionally manifold 2 if multiple bags or bottles 1 are used are then attached to a liquid inlet 116 of degasser unit 3. A degassing unit 3 of sufficient size to accommodate the volumetric flow rate desired is needed to degas the sterile fluid to the required ppm level. Degassing unit 1 is typically a hydrophobic hollow fiber or membrane filter cartridge arranged in a cross flow configuration (tangential flow) and having a molecular weight cut off (or pore size) such that only dissolved gasses pass from the fluid stream when vacuum is applied to on side of the fiber or membrane. When properly specified and used, these units can degas fluids to below 3 ppm dissolved gas at substantial flow rates. These devices are well known to the art and will not be discussed further here.

Liquid outlet tube 114 of degasser unit 3 is connected via manifold or three-way valve 4 and return tube 108 to a cap fitting 9b of reservoir container 8. Pump feed or inlet tubing 104 is likewise connected to a third port of three-way valve 4, with the other end connected to the inlet of the pump 20.

Overflow container or bag 6 is connected to a cap fitting 9a of reservoir container 8 via a respective tubing run 120. This fitting has a downcorner 122 which projects approximately half way down into container 8. Overflow container 6 must be mounted higher than rigid reservoir container 8, but lower than sterile water supply bags or bottles 1. A vent line 124 is connected between an upper end of overflow container 6 and a fourth cap fitting or connection 9d on reservoir container 8. This vent line or connection does not include a downcorner in reservoir container 8, in order to allow entrained gas to escape.

Peristaltic pump 20 is provided to force fluid through HIFU probe 11 during a surgical operation. Outlet tube 10 of pump 20 is connected to the liquid feed inlet (not labeled) of probe 11 via a tube 126. It is to be noted that the tube run including the pump inlet tube 104, an internal pump tube (not shown), the pump outlet tube 10, and the connector tube 126 may be constituted by a single unitary length of tubing.

The outlet fitting (not separately designated) of probe 11 is connected to a third opening or cap fitting 9c of reservoir container 8 via tube 106. A three way valve or stopcock 13 is provided in this tubing run 106 to accept a luer fitting of syringe 12, which may be a common off-the-shelf component.

Degasser unit 3 incorporates one or more fittings 19 for enabling connection of the degasser unit to a vacuum pump 5. In this embodiment, the degasser vacuum fittings are connected via tubing 128 to vacuum pump 5 either directly or via a manifold 130. A hydrophobic filter 14 may be installed to prevent liquid transport to vacuum pump 5 in the event of a degasser unit failure.

To begin operation, three-way valve 4 is set to flow liquid from sterile water supply bags 1 to rigid reservoir container 8. At least one shut-off valve 7 is opened, as is a pinch clamp connected to liquid outlet tube 116 of degasser unit 3. Shut off valves or pinch clamps 15 and 15a connected to tubes 120 and 124 are likewise opened. Three-way valves 4 and 13 are set to block flow to probe 11. Vacuum pump 5 is then turned on. This effectively creates a liquid charging system or hydraulic circuit 110 as shown in FIG. 4.

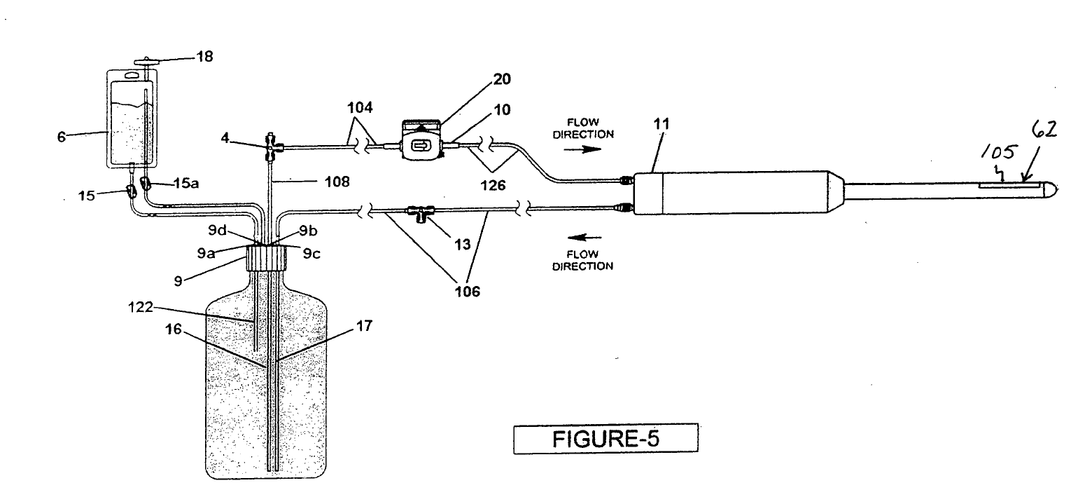

Liquid now flows under gravity head (or is alternatively pumped) through degasser unit 3. The liquid will be degassed and then flow into rigid container 8. A cap 9 of container 8 incorporates rigid downcorners 16 and 17 on fittings 9b and 9c, respectively, to effectively move the outlets of the fittings near the bottom of the reservoir container 8. Flow is maintained until container 8 is totally filled and liquid rises into overflow container 6 through downcorner 122, fitting 9a, and tube 120 by gravity head. Overflow container 6 contains a hydrophobic vent filter 18 to allow air to escape but to block liquid flow. This vent filter 18 permits air to vent from the system during filling so as to create a self air bleeding system. Once the sterile water supply bags 1 are empty or overflow bag 6 is completely full, three-way valve 4 is turned to isolate probe circuit 102 from the sterile water supply bags 1 and vacuum pump 5 is shut off. This effectively creates the liquid system as shown in FIG. 5.

Peristaltic pump 20 is subsequently activated to circulate sterile degassed water from reservoir container 8 through the tubing 108, 104, 10, and 126 into the probe head 11 and back to the rigid reservoir container via tubing 106. As the liquid is pumped, air is displaced from all of the elements and flows into reservoir container 8 and in turn rises into the overflow bag through vent line 124.

Once all of the air is expelled from probe head 11 and hydraulic circuit 102, shut-off valves 15 and 15a are turned or pinched to isolate overflow bag 6. The overflow bag is removed from the tubing.

Syringe 12 is attached to overflow bag 6 after a plunger 134 of the syringe is pushed all the way in. Overflow bag 6 is positioned such that the air is at the top and the liquid is next to the syringe connection. The syringe plunger 134 may then be retracted to fill or partially fill the syringe 12 with sterile degassed liquid without entrained air. Syringe 12 is then removed from bag 6 and attached to hydraulic circuit 102 and particularly to tube 106 via three-way valve 13. Three-way valve 13 is turned to allow liquid to flow from syringe 12 into probe outlet tube 106.

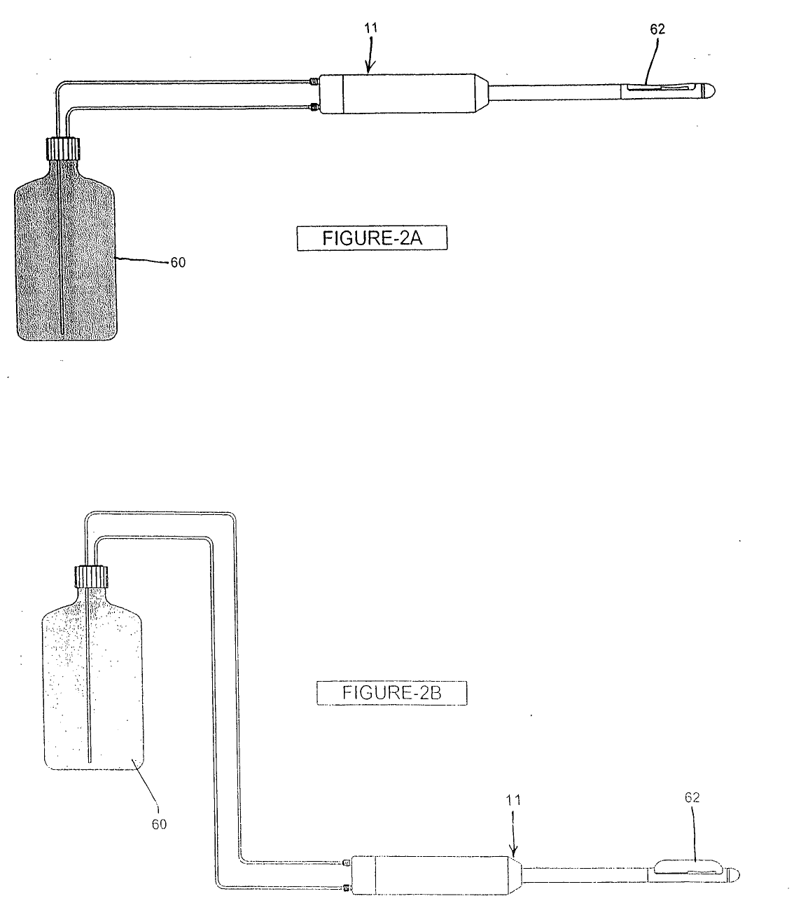

At this point, an air free, degassed and sterile liquid system exists, as shown in FIG. 6. If the peristaltic pump 20 is left on, the degassed sterile medical irrigation water will be circulated through the system and particularly through probe 11. Since the liquid system is free of compressible air and closed to the atmosphere, probe 11 may be disposed at any height relative to reservoir container 8 without causing the liquid pressure to change. This keeps the height or degree of distension of a bolus 62 (see FIG. 2B) constant.

If the bolus height is to be adjusted, the syringe plunger 134 may be moved in and out. The water in syringe 12 will serve to pressurize the liquid system. Since the bolus 62 is a flexible liquid-containing chamber or pouch, it will expand or contract as the static pressure of the system rises above the ambient air pressure. Adjusting this pressure differential with the syringe plunger 134 easily sets the amount the bolus 62 expands.

The HIFU system may then be used as per its specifications.

In this manner, a sterile, degassed supply of liquid may be manufactured on site, at relatively low cost and the tubing sets may be presterilized and disposable, reducing time and cost of the end user.

In practice, the tubing described must be manufactured with a medical grade polymer. Such polymers generally have a high surface tension that can serve to trap air bubbles or cause them to stick to the internal surfaces of the tube. This tendency can be eliminated, if desired, by coating all internal surfaces of the tubing runs and the internal surfaces of the probe assembly with an agent that reduces said surface tension and serves to effectively lubricate the surfaces to allow for quicker bubble expulsion. One such agent consists of cross-linked polymers that bond to the parent plastic and reduce surface friction or tension of the tubing. Other commercially available products can be used with equal success. This element is not mandatory to achieve the desired objectives of the invention but can serve to provide a shorter time to degas and set up a system.

Once the HIFU probe 11 and particularly bolus 62 thereof is charged with degassed liquid, the probe may be used in a tissue-necrotizing procedure wherein tissue necrosis may be induced to within a desired distance of the organ surface that the bolus engages. At the onset of the procedure, applicator surface 105 is placed in contact with an organ surface of a patient. Source or transducer 101 is energized to produce ultrasonic vibrations of a given frequency focused at a predetermined focal region inside the organ. The temperature of the liquid in bolus 62 is controlled while applicator surface 105 is in contact with the patient's organ surface to control temperature elevation in tissues of the organ between the focal region and the organ surface to necrose the tissues to within a desired distance from the organ surface.

Typically, the energizing of source or transducer 101 continues for a time period long enough to necrose tissues in the predetermined focal region. The temperature of the liquid in bolus 62 may be maintained at a preselected temperature approximately equal to a natural body temperature of the organ at least during an interval subsequent to an initial portion of the time period of focused ultrasound application, and possibly during the entire period of ultrasound application.

Alternatively, the temperature of the liquid in bolus 62 is maintained at a preselected temperature substantially equal to and less than a necrotizing temperature of the tissues of the organ (approximately 42° C.) at least during an interval subsequent to an initial portion of the period of focused ultrasound application, and in some cases during the entire period of ultrasound application.

As yet another alternative, the temperature of the liquid in bolus 62 may be increased after an initial interval of energization of ultrasound source or transducer 101. The temperature increase is typically from a first temperature substantially below the necrotizing temperature of the tissues of the organ (substantially below 42° C.) to a second temperature substantially above the necrotizing temperature (e.g. 55° C. and higher). This temperature increase may be instituted after the ultrasound application has been terminated.

A heat source or heating element 107 (FIG. 1) may be provided inside bolus 62 for regulating the temperature of the liquid inside the bolus. In addition, a temperature sensor 109 may be disposed inside or in thermal contact with the bolus for providing a temperature control unit 111 with feedback. Control unit 111 is operatively coupled with heating source or element 107 for alternately decreasing and increasing the heat output thereof in accordance with the sensed temperature of the liquid inside bolus 62 and pursuant to instructions input by a user via a keypad 113 or other interface.

In an alternative construction, a heat source or heating element 115 (FIG. 3) may be provided upstream of probe 11 and accordingly upstream of bolus 62 for regulating the temperature of the liquid inside the bolus. Temperature sensor 109 (FIG. 1), disposed inside or in thermal contact with bolus 62, provides a temperature control unit 117 with feedback. Control unit 117 is operatively coupled with heating source or element 115 for alternately decreasing and increasing the heat output thereof in accordance with the sensed temperature of the liquid inside bolus 62 and pursuant to instructions input by a user via a keypad 119 or other interface. Additionally or alternatively, control unit 117 may be connected to a pump rate controller 121 in turn operatively coupled to pump 20 for modulating the operation thereof in response to feedback from temperature sensor 109 (FIG. 1) and pursuant to instructions input by a user via keypad 119 or other interface. Accordingly, the controlling of the temperature of the liquid in bolus 62 may be implemented by controlling a rate of liquid flow through the bolus. Controlling the rate of liquid flow includes the option of at least temporarily arresting liquid flow through the supply circuit (FIG. 6) and bolus 62.

In one mode of using probe 11, the temperature of the fluid in bolus or expandable chamber 62 is typically kept below body temperature for the majority of the time of the HIFU treatment via probe 11. After a specific time, or when diagnostic visualization of the treatment zone reveals that the lesion has grown back to within the desired distance from the organ surface and concomitantly applicator surface 105 of bolus 62, the energization of source or transducer 101 is arrested or interrupted, so that the generation of HIFU energy is suspended. Then the coupling fluid temperature is increased to approximately 55 to 70° C., substantially above the necrotizing temperature of organic tissue. The bolus 62 is left in contact with the treated organ until the tissue is necrosed from the organ surface distally to the HIFU focal region, with no intervening viable tissue left. This thermally produced lesion will grow by thermal conduction from the hot bolus 62 to the original HIFU lesion.

In another mode of using probe 11, the fluid inside bolus 62 is maintained at temperature at or about body or organ temperature. This temperature may be maintained during the entire period that applicator surface 105 of bolus 62 is in contact with the target organ surface. Alternatively, this temperature may be maintained during only a portion of the time that applicator surface 105 is in contact with the target organ surface, for instance, during a terminal portion of that time.

In a further mode of using probe 11, the temperature of the fluid inside bolus 62 may be maintained just below the necrosis point of tissue, generally regarded as approximately 42° C. In this way, the heat sinking properties of bolus 62 are reduced and any heat input to the tissue from thermal conduction from the focal zone will increase the temperature of the tissue above necrosis, creating the desired contiguous lesion. Again, the temperature of the fluid inside bolus 62 may be maintained at or about the necrosis temperature during the entire period that applicator surface 105 of bolus 62 is in contact with the target organ surface. Alternatively, the temperature of the fluid inside bolus 62 may be maintained at or about the necrosis temperature during only a portion of the time that applicator surface 105 is in contact with the target organ surface, for instance, during a terminal portion of that time.

Pursuant to yet another mode of using probe 11, the temperature of fluid inside bolus 62 is controlled by temperature control unit 111 or 117 in response to feedback from sensor 109. Sensor 109 may take the form of a thermocouple, an infrared camera (external to bolus 62 but in operative contact therewith, via radiation) with minimal input from the surgical team via keypad 113 or 119. Keypads 113 and 119 are control panels that may be located on the HIFU generator user interface or remotely via an Ethernet or other electronic or radiofrequency communication link 123, 125.

Inline heating element 115 (FIG. 3), which is installed in the fluid line at the input to the HIFU probe housing, is sized to provide a temperature rise in the flowing coupling fluid to a temperature desired by the user. A feedback thermocouple (109) may be provided to communicate with the coupling fluid to monitor fluid temperature and to signal liquid temperature controller 117 to turn heating element 115 on or off or control the rate of electrical energy applied to the heating element with PID control loops in methods known to the art.

The locating of heating element 107 inside bolus 62 reduces the time lag for bolus fluid heating and also reduces the temperature loss due to heating the tubing runs throughout the probe body.

Pursuant to yet another mode of operation of probe 11, the liquid flow through bolus 62 may be stopped while the heating is taking place. In this way, minimum heat energy is needed to heat the fluid. More importantly, immediate cooling may be accomplished by turning off the heating element 107 and turning on the fluid pump 20 via pumping rate controller 121, which will flood the bolus 62 with cool fluid and allow cooling of the tissue in cases where an overheat condition is encountered or to control the lesion size and shape more precisely.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A medical procedure comprising:
providing a high-intensity focused ultrasound instrument having an applicator surface, a liquid-containing bolus acting as a heat sink, and a source of ultrasonic vibrations, said applicator surface being a surface of a flexible wall of said bolus, said source of ultrasonic vibrations being in operative contact with said bolus;
placing said applicator surface in contact with an organ surface of a patient;
energizing said source to produce ultrasonic vibrations focused at a predetermined focal region inside said organ; and
controlling a temperature of liquid in said bolus while said applicator surface is in contact with said organ surface to control temperature elevation in tissues of said organ between said focal region and said organ surface to necrose the tissues to within a desired distance from said organ surface.

2. The method defined in claim 1 wherein the energizing of said source continues for a time period and the controlling of the temperature of the liquid in said bolus includes maintaining the temperature of the liquid in said bolus at a preselected temperature approximately equal to a natural body temperature of said organ at least during an interval subsequent to an initial portion of said time period.

3. The method defined in claim 2 wherein the controlling of the temperature of the liquid in said bolus includes maintaining the temperature of the liquid in said bolus at said preselected temperature during substantially the entirety of said time period.

4. The method defined in claim 1 wherein the energizing of said source continues for a time period and the controlling of the temperature of the liquid in said bolus includes maintaining the temperature of the liquid in said bolus at a preselected temperature substantially equal to and less than a necrotizing temperature of the tissues of said organ at least during an interval subsequent to an initial portion of said time period.

5. The method defined in claim 4 wherein the controlling of the temperature of the liquid in said bolus includes maintaining the temperature of the liquid in said bolus at said preselected temperature during substantially the entirety of said time period.

6. The method defined in claim 1 wherein said instrument includes a liquid flow circuit including said bolus, the controlling of the temperature of the liquid in said bolus including controlling a rate of liquid flow through said circuit and said bolus.

7. The method defined in claim 6 wherein said instrument includes a liquid flow circuit including said bolus, the controlling of the temperature of the liquid in said bolus including at least temporarily arresting liquid flow through said circuit and said bolus.

8. The method defined in claim 1 wherein the energizing of said source continues for a time period and the controlling of the temperature of the liquid in said bolus includes increasing the temperature of the liquid in said bolus after an initial portion of said time period, the temperature increase being to a temperature substantially above a necrotizing temperature of the tissues of said organ.

9. The method defined in claim 1, further comprising terminating the energizing of said source while maintaining said applicator surface in contact with said organ surface, the controlling of the temperature of the liquid in said bolus including increasing the temperature of the liquid in said bolus after terminating the energizing of said source, the temperature increase being to a temperature substantially above a necrotizing temperature of the tissues of said organ.

10. The method defined in claim 1 wherein the controlling of the temperature of the liquid in said bolus includes heating the liquid by operating a heat source inside said bolus.

11. The method defined in claim 1 wherein said instrument includes a liquid flow circuit including said bolus, the controlling of the temperature of the liquid in said bolus including heating the liquid in a portion of said circuit upstream of said bolus and permitting the heated liquid to flow into said bolus.

12. The method defined in claim 1 wherein said instrument includes a liquid flow circuit including said bolus, the controlling of the temperature of the liquid in said bolus including at least temporarily arresting liquid flow through said circuit and said bolus.

13. The method defined in claim 1 wherein the energizing of said source continues for a time period, further comprising maintaining the temperature of the liquid in said bolus at a first temperature substantially below a necrotizing temperature of the tissues of said organ for at least an initial portion of said time period, the controlling of the temperature of the liquid in said bolus including increasing the temperature of the liquid in said bolus after said initial portion of said time period to a second temperature substantially above said first temperature.

14. The method defined in claim 1 wherein said instrument includes a temperature sensor and a heating element, the controlling of the temperature of the liquid in said bolus including automatically energizing said heating element in response to a signal from said sensor.

15. A medical treatment apparatus comprising:
a high-intensity focused ultrasound instrument having an applicator surface, a bolus acting as a heat sink, and a source of ultrasonic vibrations, said applicator surface being in thermal and operative contact with said bolus, said source of ultrasonic vibrations being in operative contact with said bolus; and
means for controlling a temperature of liquid in said bolus while said applicator surface is in contact with said organ surface to control temperature elevation in tissues of said organ between said focal region and said organ surface to necrose the tissues to within a desired distance from said organ surface.

16. The apparatus defined in claim 15, further comprising a liquid supply circuit communicating with said bolus for circulating liquid thereto, said means for controlling including a heating element disposed in said liquid supply circuit upstream of said bolus.

17. The apparatus defined in claim 16 wherein said means for controlling further includes a temperature sensor operatively connected to said heating element.

18. The apparatus defined in claim 15 wherein said means for controlling includes a heating element disposed in said bolus.

19. The apparatus defined in claim 18 wherein said means for controlling further includes a temperature sensor operatively connected to said heating element

20. The apparatus defined in claim 15, further comprising a liquid supply circuit communicating with said bolus for circulating liquid thereto, said means for controlling including an adjustable rate pump in operative engagement with said circuit.
